(19) United States
(12) Patent Application Publication
(10) Pub. No.: US 2024/0390315 A1
(43) Pub. Date: Nov. 28, 2024

(54) NUTRITIONAL COMPOSITIONS

(71) Applicant: MITOCHOLINE Ltd, London (GB)

(72) Inventors: Galina Nonina Skladtchikova, Hellemp (DK); Larisa Andreeva, London (GB)

(21) Appl. No.: 18/685,328

(22) PCT Filed: Aug. 24, 2022
(86) PCT No.: PCT/GB2022/052175

§ 371 Date: Feb. 21, 2024

(30) Foreign Application Priority Data
Aug. 25, 2021 (GB) 2112170.2

(51) Int. Cl. A61K 31/225; A23L 2/52; A61K 9/00; A61K 31/198; A61K 31/202; A61K 31/205; A61K 31/4415; A61K 31/455; A61K 31/519; A61K 31/706; A61K 31/7076; A61K 31/714; A61P 3/00

(52) U.S. Cl. CPC A61K 31/225; A23L 2/52; A61K 9/0095; A61K 31/198; A61K 31/202; A61K 31/205; A61K 31/4415; A61K 31/455; A61K 31/519; A61K 31/706; A61K 31/7076; A61K 31/714; A61P 3/00

(57) ABSTRACT

The present invention relates to nutritional compositions for maintaining and enhancing one carbon (1C) metabolism. The compositions are useful for maintaining general body health, reducing physiological consequences of metabolic stress, enhancing anabolism in a growing body or in a body recovering from a disease or trauma, activating anabolic processes in the ageing body of a mammal, such as a human individual or domestic animal. The compositions comprise a combination of choline and succinate in the molar ratio 2:1.

### CROSS-REFERENCE TO RELATED APPLICATIONS

This application is a National Stage Entry under 35 U.S.C. § 371 of International Application PCT/GB2022/052175 filed on 24 Aug. 2022, which claims priority to UK Application GB2112170.2 filed on 25 Aug. 2021, the entirety of each of the foregoing applications are incorporated herein by reference to the extent permitted by applicable patent laws and rules.

### FIELD

The present disclosure relates generally to compositions comprising a combination of choline and succinate in the molar ratio 2:1 and methods for maintaining, enhancing and restoring one-carbon (1C) metabolism, maintaining and promoting mental and physical wellbeing and general health of the body, and treating various diseases and physiological conditions associated with disbalanced 1C metabolism, e.g., diseases of the liver, joints and other organs in humans and domestic animals using the compositions.

### BACKGROUND

Normal functioning of one-carbon (1C) metabolism which comprises a series of interlinking metabolic pathways that include the methionine and folate cycles that are central to cellular function, providing 1C units (methyl groups) for the synthesis of DNA, polyamines, amino acids, creatine, and phospholipids. The source of the methyl group in the body cells are methyl donor molecules, including methionine, folate, betaine, and choline, which could be either supplied via diet or synthesized by the cells.

A choline-rich diet is a good source of methyl groups. Food supplementation with choline has been proven to be beneficial for the health of both humans and domesticated mammals. However, there is still a lack of commercial choline-containing food products and dietary supplements that could support and enhance both one-carbon (1C)) and energy metabolisms simultaneously. In view of the facts that almost every country of the world at present is experiencing a shift in the distribution of a country's population towards older ages, people of all ages live under increasing press of the metabolic stress leading to various diseases, and there are huge socio-economic problems resulting from the effects of an environmental and psychological pressure, it is a great need in agents, that are safe and effective dietary supplements that could support, enhance and restore body and mental health and delay delaying ageing processes by preventing and diminishing negative consequences of metabolic and psychological stress affecting one-carbon (1C) and energy metabolism.

### SUMMARY

A first aspect of the disclosure relates to a composition comprising choline and succinate in the molar ratio 2:1, for use in supporting, enhancing, and/or restoring one-carbon (1C) metabolism in a mammal, such as a human subject or domesticated animal, like a cattle or pet animal. Advantageously, the composition may further comprise nicotinamide (NAM), or nicotinamide riboside, wherein the molar ratio choline: succinate: NAM/nicotinamide riboside is from about 2:1:0.001 to about 2:1:10, at least one vitamin B selected from the group consisting of vitamin B12 (cobalamin or methylcobalamin), vitamin B6 (pyridoxine), and vitamin B9 (folic acid or folacin), and/or docosahexaenoic acid (DHA).

A second aspect of the disclosure relates to a method for the dietary management and/or mitigation of the risk of
metabolic stress, catabolism and/or increased energy expenditure due to injury, Illness and/or unbalanced nutrition; and/or
a condition or a symptom associated with an imbalanced, damaged or reduced one-carbon (1C) metabolism; or
occurrence and/or re-occurrence of a symptom or condition associated with an imbalanced, damaged or reduced one-carbon (1C) metabolism,

in a mammal subject, comprising administering to said human at least once a day a composition comprising choline and succinate in the molar ratio 2:1, and/or (i) NAM, or nicotinamide riboside, wherein the molar ratio choline: succinate: NAM/nicotinamide riboside is 2:1:0,001-10; (ii) least one vitamin B selected from the group consisting of vitamin B12 (cobalamin or methylcobalamin), vitamin B6 (pyridoxine), and vitamin B9 (folic acid or folacin), and/or (iii) docosahexaenoic acid (DHA).

A third aspect of the disclosure relates to a dietary supplement, food or beverage product comprising di-choline succinate and docosahexaenoic acid (DHA), wherein the amount of di-choline succinate is from around 10 mg to around 5000 mg, and the amount of DHA is from around 200 mg to around 1000 mg. The dietary supplement, food or beverage product may further advantageously comprise nicotinamide, or nicotinamide riboside, wherein the molar ratio choline: succinate: nicotinamide/nicotinamide riboside is from around 2:1:0.01 to around 2:1:10, and/or least one vitamin B selected from the group consisting of vitamin B12 (cobalamin or methylcobalamin), vitamin B6 (pyridoxine), and vitamin B9 (folic acid or folacin).

Subjects who would benefit from intake of compositions of the invention may be any human subjects, such as healthy human subjects with or without particular demand for improving their diet; metabolically and/or psychologically weakened individuals, such as ageing individuals and/or individuals who are weakened due to disease, injury, unbalanced diet or certain mental conditions, such as e.g., psychological stress, fatigue, insomnia or mental depression, or environmental factors, like socio-economical pressure, etc. Further, compositions of the disclosure in some examples are formulated and beneficially used as a supplement to animal food, including both cattle and pet animal food.

### DETAILED DESCRIPTION

### Introduction

In the body, choline is oxidized to betaine (N,N,N-trimethylglycine), which is a major source of methyl groups, by the mitochondrial enzymes, choline dehydrogenase and betaine aldehyde dehydrogenase, which are expressed in the cytosol and mitochondria. Betaine contains three methyl groups, of which one is donated to homocysteine and the other two are used to synthesize amino acid glycine that is used further in synthesis of many important molecules, such as bile acids, proteins, creatine, etc. Low concentrations of plasma betaine were strongly correlated with an increased concentration of plasma homocysteine, which is a biomarker of a decreased rate of methylation processes resulting in various diseases and cell ageing due to lack of methyl groups or methyl group donors in the body. Increased amounts of homocysteine were associated with numerous pathological conditions, e.g., kidney dysfunction, CVD, ischemic stroke, and neurological problems like autism, Parkinson's disease, Alzheimer's disease, and seizures. Homocysteine also plays a role in oxidative stress, enhancing the production of reactive oxygen species, and hence is one of the causes of lipid peroxidation and cell membrane injury. Hypomethylation is also involved in energy metabolism disorders.

The role of betaine and choline in energy metabolism appears to be beyond their effect on gene methylation since folate, another donor of the methyl group in methylation processes of the body, is not known to show the same effect on energy metabolism, e.g., betaine improves conditions of nonalcoholic fatty liver and associated hepatic insulin resistance, and is a positive regulator of mitochondrial respiration.

Choline, in a form of a salt of succinic acid (di-choline succinate), has been shown to be a potent sensitizer of the neuronal insulin receptor. Further, the inventors of the present disclosure have shown in copending applications that compositions of dicholine succinate with nicotinamide are synergistically effective for increasing the levels of NAD, ATP, and phosphocreatine in the brain cells (WO2019002858).

Succinate, which a part of dicholine succinate salt, is a major intermediate of the tricarboxylic acid cycle (TCA), that interacts directly with the mitochondrial electron transport chain (ETC), enabling a ‘shortcut’ route to ATP production via oxidative metabolism. Further, it has been demonstrated that succinate potentiates choline transport into the mitochondria via a specific choline transporter by increasing the mitochondrial membrane potential, thereby indirectly increasing the cellular levels of betaine available for methylation processes of the body.

Availability of methyl groups is crucial for normal functioning of the body, and the demand is particularly high during the body growth, tissue regeneration, intense physical or mental activity, in aging or disease. Body processes, both anabolic and catabolic, also demand constant energy supply in the form of energy molecule ATP and NAD that are synthesized in mitochondria during respiration. There is a need for nutritional compositions capable of maintaining and increasing energy levels and supporting supply of methyl groups used in synthetic processes for extended periods of time to prevent deterioration of the body and slowing down body aging.

Furthermore, the inventors have surprisingly found that choline (cation) and succinate (anion 2−) combined in the molar ratio around 2:1 supports one-carbon (1C) metabolism in humans better than existing choline dietary supplements for 1C metabolism, such as for example, choline bitartrate or choline chloride. Advantageously, a combination of choline (cation) and succinate (anion 2−) combined with nicotinamide (NAM), wherein a molar ratio of choline: succinate: NAM is from about 2:1:0,001 to about 2:1:10, can enhance mitochondrial function working synergistically both to boost production of “energy” molecules ATP and NAD and to provide methyl groups (1C groups) needed for diverse synthetic processes in body cells.

These multiple synergetic results of the later composition may further be extended, where the composition further comprises an omega-3 unsaturated acid, such as for example, docosahexaenoic acid (DHA), preferably DHA. Utilization of dietary DHA in the body is more efficient when it is combined with a choline dietary supplement. The synergetic effects of the compositions are greatest when choline (cation) and succinate (anion 2−) are derived from dicholine succinate salt (DiSu) present in the composition, compared to other sources of choline and succinic acid. However, an enhancement of both 1C metabolism and energy molecule production may also be achieved when other salts of the choline and succinic acid are the source of choline and succinate, albeit to a lesser degree. Yet, neither single compounds nor combinations of only two of the three compounds of the above composition of the disclosure were able to achieve the double effects of enhanced generation energy and 1C metabolites.

Without being bound to a theory, it is thought that compounds of the composition, choline, succinate and NAM, present in a molar ratio within the range of 2:1:0,001 to 2:1:10, work synergistically in a way that the succinate anion of the composition increases mitochondrial membrane potential, which facilitates influx of choline into mitochondrion, where it is oxidized to betaine; at the same time, NAM and succinate get involved in the chain of reactions within the mitochondrion that lead to synthesis of NAD and ATP molecules, which, together with betaine, are transported into cytosol and utilized in the chain of the interconnected reactions of 1C metabolism in the body. As addition to increased levels of betaine, ATP, and NAD in cells of the body, a synergetic effect of compositions also influence the levels of S-adenosylmethionine (SAMe) and homocysteine, reflected in an increase of SAMe and decrease in homocysteine.

The inventors found that an oral intake of a composition consisting essentially of DiSu and NAM, wherein the ratio of choline: succinate: NAM is about 2:1:0,001-10, by human individuals during about 1 week to about 4 weeks, is associated with at least one of the following physiological effects: increased skeletal muscle strength, tone and endurance, and quick recovery from extensive physical or mental exercise, i.e., a minimal or no feeling of exhaustion or tiredness following the exercise, that typically would take significantly longer time to achieve and would require a good external supply of “energy”, in form of glucose, and, one-carbon metabolism supply, in form of protein building blocks amino acids, to restore exhausted internal resources. Further, a daily intake of the composition has other beneficial effects, e.g., an increased general feeling of well-being, positive thinking, and motivation.

Accordingly, the subject matter of the disclosure relates to compositions that are effective to support 1C metabolism, e.g., to maintain, enhance, or restore 1C metabolism in mammals, such as human and domesticated animal subjects. The compositions comprise at least two compounds, choline (cation) and succinate (anion 2−) in the molar ratio around 2:1, in some implementations, at least three compounds choline (cation) and succinate (anion 2−) and nicotinamide (NAM) in a molar ratio within the range from about 2:1:00,1 to about 2:1:10. Non-limiting further implementations of compositions of the disclosure and their uses are described in detail below.

All terms and definitions explained throughout the specification of the disclosure relate to all aspects and implementation of the disclosure, unless otherwise specified.

The term “One-carbon (1C) metabolism” means a series of interlinking metabolic pathways that include the methionine and folate cycles that are central to cellular function, providing 1C units (methyl groups) for the synthesis of DNA, polyamines, amino acids, creatine, and phospholipids.

The wording “composition consisting essentially of”<named compounds>means that the named compounds of the composition are material for the claimed biological effect associated with the composition. Examples of such compounds are described in the specification of the disclosure.

The term “synthetic” in the present context means a man-made composition. The compositions of the disclosure, in typical examples, comprise synthetically prepared molecules that are structurally identical to the molecules that naturally occurring in cells of the living bodies, and, in some examples, comprise artificial molecules that do not have natural structural equivalents.

The term “about” means a deviation from the indicated value by 0.01% to 10%, such as from 0.5% to 5%.

The term “choline” (“choline cation”) means a compound having the chemical formula C₅H₁₄NO⁺ (CAS No. 62-49-7). According to the present disclosure, the choline compound of compositions described herein is typically derived either from choline hydroxide, or a salt of choline, e.g., choline bitartrate, choline chloride, dicholine succinate. However, Other water soluble compounds that can provide choline cation are also contemplated.

The term “succinate” means the divalent succinic acid anion having the chemical formula C₄H₄O₄⁻² (CAS No. 110-15-6). According to the present disclosure, the succinate compound of compositions described herein is typically derived either from succinic acid or a divalent salt of succinic acid, e.g., di-sodium succinate or di-choline succinate. However, other water soluble compounds that can provide divalent succinate anion are also contemplated.

The terms “dicholine succinate”, “choline succinate salt (2:1)” and “DiSu” are interchangeable and mean the molecule of formula (I) (CAS No. 109438-15-5):

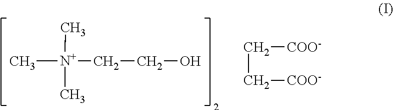

The term “nicotinamide” or “NAM” means the molecule identified with CAS No. 98-92-0.

The term “derivate of nicotinamide” or “NAM derivate” means a molecule that is derived from NAM by a synthetic process, i.e., NAM is a starting molecule for the synthesis of the derivate such as nicotinamide riboside (CAS No: 1341-23-7) or nicotinamide mononucleotide (CAS No: 1094-61-7). The preferred NAM derivate is nicotinamide riboside.

The term “mammal subject” in the present context means a human individual or a domesticated mammal such as a dog, cat, rabbit, cattle, pig, goat, sheep, horse, etc.

The term “metabolic stress” denotes a condition in which the body metabolizes nutrients at a greater rate than the nutrients are supplied to the body, which can result in a state of destructive metabolism, also herein referred to as catabolism. This state can be induced by illnesses, infections, mental stress, disproportionate physical exercise, etc., which are often accompanied by lack or excess of nutrients in the diet. This state may also be caused by surgery or harsh therapeutic treatment, e.g., radiation or chemotherapy, which is disruptive of normal metabolism processes. Further the state can be induced by physical and psychological traumas which induce a necessity for high caloric intake. For example, a burn patient may require as many as about 7,000 calories per day due to damage to the body and the results thereof occasioned by the burn.

Patients suffering from a catabolic state often suffer from reduced anabolism. The term “anabolism” also called biosynthesis, means the sequences of enzyme-catalyzed reactions by which relatively complex molecules are formed in living cells from nutrients with relatively simple structures. In growing cells, anabolic processes predominate over catabolic ones whereas, in nongrowing cells, a balance exists between the two. Under metabolic stress, catabolism prevails over anabolism. The catabolic state often results in severe body weight loss which can result in pronounced complications to the primary malady, severe body damage and even death. Catabolic state is also characterized by disbalanced energy metabolism, which is often reflected by excessive accumulation of fat in the body, fatigue, mental depression, decreased cognitive capacity, etc.

Although catabolism may be induced in the aforementioned mammal subjects by a shortage of nutrient intake, insufficient net intake of methyl donors is believed to also be a very important factor, in order for mammals to be able to effectively metabolize nutrients needed for anabolic processes, a surplus of methyl donors is required. In mammals undergoing metabolic stress due to injury, trauma, infections, etc. the net requirement for methyl donors is exceptionally high because methyl donors are excreted and/or metabolized at an elevated rate and/or because anabolic processes are occurring at an extremely high rate. The average diet does not provide methyl donors in a net amount that is sufficient for preventing or treating catabolism in such individuals. Consequently, supplementation of methyl donors, optionally together with supplementary molecules, like co-factors of the essential metabolic enzymes, e.g., vitamin B12 (cobalamin or methylcobalamin), vitamin B6 (pyridoxine), is highly desirable in mammal subjects suffering from metabolic stress due to injury, trauma, infection, an unbalanced diet, or physical or mental exercise overload, disease or aging.

Human individuals who would especially benefit from food or food supplements comprising/essentially consisting of compositions of the present disclosure are those who are suffering or recovering from chronic inflammatory diseases and conditions, including Crohn's disease, Inflammatory Bowel Disease (IBD), degenerative diseases of neural system, e.g., Alzheimer's, Parkinson's, Haddington's diseases; musculoskeletal diseases, such as muscle-wasting, muscle degenerative disease, myopathies, age-related decline in muscle function, frailty, pre-frailty, neuromuscular diseases, Duchenne muscular dystrophy, sarcopenia, muscle atrophy and/or cachexia, muscle loss, a muscle function disorder, age-related decline in muscle function, age-related sarcopenia, age-related muscle-wasting, physical fatigue, muscle fatigue, inclusion body myositis, sporadic inclusion body myositis. In one embodiment, a human subject is an individual who is diagnosed with a metabolic myopathy, such as acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5); lever diseases, especially, Non-Alcoholic Fatty Liver Disease (NAFLD). These human individuals are considered as target groups for dietary management of their conditions with compositions of the disclosure. In some implementations, the dietary management of physiological conditions associated with metabolic stress and/or unbalanced 1C metabolism with use of compositions of the disclosure may be preferred.

The term “dietary management” in the present context means a practice of providing nutritional options for an individual with health concerns through supervision and optimization of the individual diet to target nutritional deficits and/demands instead of therapeutic intervention. The dietary management practice does not substitute a required therapeutic treatment, if it is required, but enhance its efficiency and thereby facilitates the patient recovery. In generally healthy individuals that have unbalanced diet, overexercise (both mental and physical), weakened by disease, therapy or injury, stressed due to hazardous environmental factors, etc., the dietary management works as preventive and supportive means allowing individuals to avoid developing or enhancing their pathologic conditions. The dietary management includes the steps of selecting particular nutrients for the diet that would help to an individual to combat consequences of their hazardous physiological conditions, and of treating the individual with the selected nutrients by including them into the individual's diet in a form of nutritional supplement(s), food or beverage fortifications, including medical foods and beverages.

Accordingly, compositions of the disclosure may advantageously be taken daily as a dietary supplement, e.g., by
(i) an undernourished individual or an individual receiving an unbalanced diet, e.g., a diet with insufficient amount of folate, and/or choline;
(ii) an overweight or obese individual;
(iii) an individual undergoing or recovering from metabolic, psychological and/or environmental stress;
(iv) an aging individual, such as a 50+ years old individual, especially such as a 75+ years old individual, or an elderly individual suffering from a severe body weight loss;
(v) a patient undergoing or recovering from a therapeutic treatment;
(vi) a patient having a chronic, degenerative or inflammatory disease; and/or
(vii) an individual who is recovering from a surgery or physical trauma.

Other human subjects that may especially benefit from intake of the present compositions include patients undergoing radiation therapy or chemotherapy; trauma patients or patients who have undergone surgery; individuals suffering from a dysfunction of the gastrointestinal tract; pregnant or lactating females; infants, especially pre-term infants, and athletes.

Further, advantageously, the present composition has also been found beneficial for:
enhancing anabolic processes as it is strongly desired in pregnant woman and infants, especially those of low birth weight;
increasing the lean body mass, and decreasing accumulation of excessive fat leading to overweight and obesity;
preventing and/or treating the muscle catabolism and/or cachexia that may occur due to or following surgery, injury, cardiovascular disease, pancreas disorders, tumors, especially liver, pancreas, lung and kidney tumors, malnutrition, neurological disorders, lung emphysema and other respiratory diseases, liver disorders like cirrhosis, severe inflammation like during inflammatory bowel disease, pancreatitis, hepatitis, AIDS and during severe insulin resistance as may occur in diabetes;
improving the energy status of body tissues and cells.

Advantageously, the increase of lean body mass and energy status leads to an increase in muscle strength and power.

In addition, vegetarians and vegans will profit from compositions of the present disclosure since presence of creatine in vegan/vegetarian diet is marginal. Compositions of the present disclosure comprising choline will serve as a source of creatine for these individuals, since creatine is synthesized in the body from glycine which is a product of oxygenation of choline. In some examples, compositions of the present disclosure may comprise a nutritionally recommended amount of creatine.

Accordingly, in one example, the present disclosure provides a composition containing methyl donor, choline, and energy metabolism enhancing compounds, succinate and NAM, wherein the molar ratio of choline: succinate: NAM is from about 2:1:0,001 to about 2:1:10, which composition may be advantageously used for stimulating and/or enhancing anabolic processes and/or increasing of the lean body mass, and/or preventing and/or treating muscle catabolism or cachexia, and/or improving the energy status of tissues and cells, wherein the composition is formulated for an oral administration to a mammal subject, such as a dietary supplement, food or beverage, or food or beverage fortifier.

Compositions of the present disclosure are also efficient as animal food supplementation, in particular, for feeding cattle and or other domestic animals or pets like dogs, cats, rabbits, etc.

For example, in pets, compositions of the present disclosure may be used as nutritional supplements or medical foods helping to prevent or treat seizures and cognitive disorders, liver and gallbladder disease, especially those resulting from fat accumulation in the liver (fatty liver disease called hepatic lipidosis). Pets with high levels of blood cholesterol (as seen in dogs with hypothyroidism and in the genetic disorder hyperlipidemia in Miniature Schnauzers) may also respond to choline supplementation comprised in the present compositions. In domestic animals, like cattle, compositions of the present disclosure may effectively reduce fatty lever syndrome and ketosis. Currently, a major choline food supplement for domestic animals and pets is choline chloride. However, high amounts of choline chloride may be toxic. A synergetic salt of choline, dicholine succinate (DiSu), comprised in some examples of the composition of the present disclosure may be advantageously substituted for choline chloride due to more efficient utilization of choline in 1C metabolism facilitated by succinate, which itself is an essential metabolite, and removal of toxic chloride from the diet.

### Non-Limiting Examples of Compositions of the Disclosure.

The present disclosure relates to compositions comprising, (in some implementations, consisting essentially of) choline (cation) and succinate (divalent anion); preferably the choline and succinate are present in the molar ratio about 2:1, preferably, said choline and succinate are present in compositions of the present disclosure in the form of dicholine succinate salt (DiSu), i.e., DiSu is a part of the composition. Alternatively, the choline cation of the composition may derive from another salt of choline, e.g., choline bitartrate (CAS No. 87-67-2), and succinate anion may derive from another salt of succinic acid, e.g., succinic acid disodium salt (CAS No. 6106-21-4). Compositions consisting essentially of choline bitartrate or choline fumarate, and succinic acid disodium or diammonium salt may be preferred in some examples of the present disclosure. The molar ratio of choline and succinate in such compositions is about 2:1.

Advantageously, a composition of the present disclosure comprising choline and succinate (molar ratio 2:1) comprises NAM or a NAM derivate. The amount of NAM, or a NAM derivate, preferably nicotinamide riboside, in compositions of the present disclosure may vary within the range of molar ratio of choline to succinate to NAM/NAM derivate from about 2:1:0.01 to about 2:1:10. Compositions with a molar ratio of the individual compounds within this range act synergistically and significantly enhance both mitochondrial function in body cells including increasing the cellular production of major energy molecules ATP and NAD and generation of methyl groups. As discussed herein, these compositions are effective in dietary management of symptoms and conditions associated with imbalanced or weakened 1C metabolism, in conditions of metabolic stress, in conditions demanding increasing the rate and efficiency of anabolic processes, like in a growing body or body recovering from different stressful physiological or mental conditions. The compositions are beneficial as nutritional food supplements to improve physical, mental and metabolic health both in generally healthy physically active human subjects and in human subjects that are physically weaken due to ageing or a disease, an injury, or an imbalanced diet, i.e., a diet that lacks or has an excess of nutrients.

To obtain the above effects, when used for the purposes described herein, the essential compounds of the composition, i.e., choline (cation), succinate (divalent anion) and, advantageously, NAM, or a NAM derivate, are present in so called “effective amounts”. The effective amounts of the compounds may vary depending on the aim and/or method of use, and on the subject in need. These implementations are discussed below and exemplified by non-limiting working examples.

In some examples, a composition of the present disclosure comprising choline, succinate and NAM may further comprise other compounds that could beneficially enhance the effect of compositions on 1C metabolism and recovery of the body from metabolic stress. For example, vitamins B, such as vitamin B12 (cobalamin or methylcobalamin) and/or vitamin B6 (pyridoxine), are co-factors of essential enzymes involved in 1C metabolism. Accordingly, in some examples, compositions of the present disclosure may further comprise one or both vitamin B12 and vitamin B6, and/or vitamin B9. In other examples, creatine, or a creatine precursor, such as e.g., amino acids glycine and arginine, may also be part of a composition of the present disclosure. In one embodiment, a composition comprising other useful additives to a composition comprising choline, succinate and/or, NAM, or a NAM derivate, are discussed below. In some preferred examples, the compositions comprising choline, succinate, and/or NAM, may comprise an omega-3 unsaturated acid, such as eicosapentaenoic acid (EPA) and/or docosahexaenoic acid (DHA).

Compositions of the present disclosure may be formulated as nutraceutical, nutritional, or pharmaceutical compositions. These formulations are to comprise effective amounts of the effective ingredients of the composition of the present disclosure, and in an appropriate molar ratio as described above. The different formulations can be prepared according to standard rule and proceeding established in the corresponding art.

The term “nutraceutical” means a pharmaceutical-grade standardized nutrient. The term “pharmaceutical” in the present content means a pharmaceutical grade compound prescribed as medicament to treat a disease. The term “nutrient” means in the present context substance that provides nourishment essential for the maintenance of life of a human. The term “nutritional” in the present context means that the composition is for the dietary supplementation of a human individual. The term “dietary supplement” means a product taken by mouth that contains a dietary ingredient, e.g., a nutrient, intended to supplement the diet.

The amounts of choline and succinate, e.g., DISU, NAM, DHA, and other compounds, in a composition of the present disclosure may be adjusted for use by a particular individual or a group of individuals according to the individual's needs, age, physiological conditions, etc., and depending on the dosage form and administration regime For example, the amount of DiSu per serving may vary from 10 mg to 1000 mg per serving, and it can be served in one or more dosages a day. The amount of NAM in the composition may vary from 10 mg to 4000 mg per serving, served as one or more dosages a day, such as about 25-2000 mg per serving, served as several dosages per day, or about 50-1000 mg per serving served as several dosages per day, etc., wherein the daily dose of NAM will depend on the dietary demand of a concrete human individual or a group of human individuals. The amount of DHA may vary from around 200 mg to around 1000 mg per dosage. Non-limiting working examples of dietary compositions are described in Examples. A daily intake of about 4000 mg of NAM in the composition is considered safe and effective for any described herein purpose. According to the present disclosure, an individual may intake a composition comprising up to 4000 mg NAM, or a NAM derivate, and up to 1000 mg DISU, or the corresponding amounts of choline and succinate derived from other salts of choline and succinic acid, daily without having any side effects. In one preferred embodiment, a composition of the present disclosure is a nutritional composition and comprises essentially DiSu and NAM, wherein the molar ratio of choline, succinate and nicotinamide in the composition is about 2:1:0.4, correspondingly. In another preferred implementation, the molar ratio of choline cation, succinate anion (2−) and nicotinamide in the composition is about 2:1:1. The term “about” in the present context means a 1-10% deviation from the indicated value, e.g., the choline: succinate ratio in a composition of the present disclosure may vary between 1.7:1 and 2.3:1.

In some examples, when big domestic animals, like dairy cattle, are concerned, compositions of the present disclosure may comprise up to 15-20 g choline cation in the form of choline succinate (2:1) salt.

Preferably, intake of compositions of the present disclosure is continuous for a period of several days (2-6 days), preferably, at least one week or, more preferably, for a longer period, such as 2 to 4 weeks, 1 to 12 months or longer. There is no limitation for how long the composition can be administered as a dietary supplement. The intake can be interrupted at any time and resumed again when the individual feels that it is needed, e.g., in connection with changes in individual's lifestyle, health, individual's physical/mental conditions, or age. A dietary manager of ordinary skill can readily determine the amounts of ingredients of a dietary composition of the present disclosure according to the accepted rules and regulations.

As mentioned above, in some examples, the subject matter of the disclosure relates to nutritional compositions comprising additional nutrients. Such nutritional compositions may be in the form of any nutritional product including, without limitation, a food, a beverage, a dietary supplement, a functional food, and a medical food. In one preferred example, a composition of the disclosure is an aqueous nutritional composition, e.g., a drink or beverage, such as a sport beverage capable of enhancing anabolism. A sport nutritional supplement, food, or beverage is one of various preferred examples of a nutritional composition of the disclosure. In one preferred example, a sport nutritional supplement, food, or beverage, comprises from around 10 mg to around 5000 mg of a composition consisting essentially of choline and succinate in a molar ratio of about 2:1. In another example, a sport nutritional supplement, food or beverage comprises from around 10 mg to around 5000 mg of a composition consisting essentially of choline, succinate, and nicotinamide, or a nicotinamide derivate, in the molar ratio from around 2:1:0.01 to around 2:1:10. Preferably, choline and succinate are present in a sport nutritional supplement, food or beverage of the disclosure in the form of dicholine succinate salt. A sport nutritional supplement, food or beverage of the disclosure is useful for maintaining, improving, or restoring physical performance, muscular strength and/or muscular endurance in a human subject who is using significant time on physical exercise to improve his/her physical performance and increase the muscle mass.

In one example, a composition comprising from around 10 mg to around 5000 mg and consisting essentially of choline and succinate in the molar ratio about 2:1, or a composition comprising from around 10 mg to around 5000 mg and consisting of choline, succinate and nicotinamide, or a nicotinamide derivate, in the molar ratio from around 2:1:0.01 to around 2:1:10 may be advantageously used as a supplement to ketogenic diet, e.g., included in a ketogenic drink or food product. Elevated circulating ketone bodies from ketogenic diet used by skeletal muscle as a fuel alter substrate competition for respiration, while improving oxidative energy transduction under certain conditions, such as endurance exercise. Consequently, combination of compositions of the present disclosure with nutritional ketosis may help to unlock greater human metabolic potential, e.g., in endurance exercise.

Compositions of the present disclosure that comprise choline, succinate and nicotinamide, or a nicotinamide derivate, in the molar ratio from around 2:1:0.01 to around 2:1:10, may be advantageously used in treating or preventing metabolic stress, and/or reducing catabolism and/or for stimulating anabolism in subjects in need, such as a human or domesticated animal, who affected by lack or excess of nutrients in the diet, and/or has an increase in energy expenditure due to injury or illness. Such compositions can help with both normalization or optimization of one-carbon metabolism and enhancement of cell energy metabolism by synergistically supporting generation of ATP, NADH and FAD in mitochondria.

In practicing the examples disclosed herein, the compounds of the composition can be prepared by any process known in the art or obtained from a known commercial manufacturer, e.g., nicotinamide or its derivatives, choline bitartrate, succinate disodium salt, may be obtained from Merck. DiSu can be prepared by the reaction of choline hydroxide (CAS No. 123-41-1) with succinic acid (CAS No. 110-15-6) as, e.g., described in WO2009/022933A1.

The nutritional compositions described herein may be prepared by procedures well-known from the art and may contain some other optional ingredients. Such optional ingredients generally are used individually at levels from about 0.0005% to about 10.0% by weight of the composition. Examples of suitable optional ingredients include, but are not limited to, carriers, minerals, carbohydrates, lipids, vitamins, co-factors, buffers, flavors and sweeteners, inorganic salts, cations, and anions typically abandoned in natural drinking water, taste modifying and/or masking agents, carbon dioxide, amino acids, organic acids, antioxidants, preservatives, and colorants.

The nutritional compositions can be combined with one or more carriers and used in the form of ingestible tablets, buccal tablets, troches, capsules, elixirs, suspensions, syrups, wafers, chewing gums, foods, beverages, and the like.

Non-exclusive examples of ingredients which can serve as carriers include water; sugars, such as glucose, lactose, and sucrose; cellulose, and its derivatives; starches, such as corn starch and potato starch; powdered tragacanth; malt; gelatin; talc; excipients, such as cocoa butter; oils, such as olive oil, peanut oil, cottonseed oil, corn oil and soybean oil; glycols, such as propylene glycol; esters, such as ethyl oleate and ethyl laurate; polyols, such as glycerin, mannitol, sorbitol, and polyethylene glycol; agar; buffering agents; water; pH buffered solutions; and other non-toxic compatible substances employed in formulations. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Non-exclusive examples of antioxidants are Vitamin E, ascorbic acid, carotenoids, aminoindoles, Vitamin A, uric acid, flavonoids, polyphenols, herbal antioxidants, melatonin, lipoic acids, and mixtures thereof.

Non-exclusive examples of useful inorganic salts typically abandoned in natural drinking water are sodium carbonate, sodium bicarbonate, potassium chloride, magnesium chloride, calcium chloride, and mixtures thereof.

Non-exclusive examples of useful cations are sodium, potassium, magnesium, calcium, zinc, iron, and mixtures thereof.

Non-exclusive examples of useful anions are fluoride, chloride, bromide, iodide, carbonate, bicarbonate, sulfate, phosphate, and mixtures thereof.

Non-exclusive examples of suitable buffers are phosphate buffer, glycine buffer, citrate buffer, acetate buffer, carbonate buffer, tris-buffer, triethanolamine buffer, and succinate buffer.

Non-exclusive examples of suitable flavors are synthetic flavor oils; flavoring aromatics and naturals oils such as cinnamon oil, oil of wintergreen, peppermint oils, clove oil, bay oil, anise oil, eucalyptus, thyme oil, cedar leave oil, oil of nutmeg, oil of sage, oils of citrus fruits, oil of bitter almonds, and cassia oil; plant extracts, flowers, leaves, fruits, vanilla, chocolate, mocha, coffee, apple, pear, peach, citrus such as lemon, orange, grape, lime, and grapefruit; mango, strawberry, raspberry, cherry, plum, pineapple, and apricot, and combinations thereof.

Non-exclusive examples of suitable sweeteners are natural and synthetic sweeteners. Non-exclusive examples of natural sweeteners are naturally occurring substances, sucrose, extracts from naturally occurring substances; extracts of the plant Stevia rebaudiana Compositae Bertoni such as stevia, steviol, rebaudiosides A-F, and dulcosides A and B; extracts of Thladiantha grosvenorii such as mogroside V and related glycosides and triterpene glycosides; phyllodulcin and its derivatives; thaumatin and its derivatives; mogrosides such as mogroside IV, mogroside V, siamenoside, and mixtures thereof; genus Siraitia including S. grosvenorii, S. siamensis, S. silomaradjae, S. sikkimensis, S. Africana, S. borneesis, and S. taiwaniana; naturally-occurring glycosides; and active compounds of plant origin having sweetening properties, and mixtures thereof. Non-exclusive examples of synthetic sweeteners are aspartame saccharin, and mixtures thereof.

Non-exclusive examples of suitable colorants are dyes suitable for food such as those known as FD&C dyes, natural coloring agents such as grape skin extract, beet red powder, titanium dioxide, and beta-carotene, annatto, carmine, chlorophyll, paprika, and mixtures thereof.

Non-exclusive examples of useful organic acids are acetic acid, butyric acid, malic acid, pyruvic acid, glutamic acid, citric acid, omega-3 unsaturated acids, linoleic acid, linolenic acid, eicosapentaenoic acid, docosahexaenoic acid, aspartic acid, and mixtures thereof.

In one preferred embodiment, a composition of the present disclosure an omega-3 unsaturated acid, such as eicosapentaenoic acid (EPA) and/or docosahexaenoic acid (DHA), preferably, DHA.

Non-exclusive examples of useful amino acids are glycine, arginine, L-tryptophan, L-lysine, methionine, threonine, Levocarnitine, and L-carnitine.

Non-exclusive examples of useful vitamins are thiamin, riboflavin, nicotinic acid, panthothenic acid, biotin, folic acid, pyridoxine, vitamin B12, lipoic acid, vitamin A, vitamin D, vitamin E, ascorbic acid, choline, carnitine; alpha, beta, and gamma carotenes; vitamin K, and mixtures thereof.

In one preferred example, compositions of the present disclosure comprises one or more vitamins B, preferably, the vitamin B is selected from the group consisting of vitamin B12 (cobalamin or methylcobalamin), vitamin B6 (pyridoxine), and vitamin B9 (folic acid or folacin).

Non-exclusive examples of useful co-factors are thiamine pyrophosphates, flavin mononucleotide, flavin adenine dinucleotide, pyridoxal phosphate, biotin, tetrahydrofolic acid, coenzyme A, coenzyme B12, coenzyme B6, 11-cis-Retinal, 1,25-dihydroxycholecalciferol and mixtures thereof.

In one embodiment, a nutritional composition of the disclosure may comprise compounds that are able to increase the blood circulation, e.g., an extract of Ginkgo biloba or ginseng. In some examples, a composition of the disclosure may comprise an antioxidant, e.g., Astaxanthin, resveratrol, flavonoids.

As mentioned, the nutritional compositions can be used as a component of dietary supplement, a food product or a beverage.

Nonexclusive examples of food products include regular foods, beverages, and medical foods.

The term “medical food” refers to a food which is formulated to be consumed or administered enterally under the supervision of a physician and which is intended for the specific dietary management of a disease, condition, or disorder.

Preferably, the nutritional compositions are administered to a human orally for a period of at least one day or, preferably, longer (as discussed above).

Non-limiting preferred implementation of compositions, dietary supplements, food or beverage products, and/or methods relating to the subject matter disclosed herein are described in the clauses below:
1. A composition comprising choline and succinate in the molar ratio about 2:1, for use in supporting, enhancing and/or restoring one-carbon (1C) metabolism in a mammal, such as a human subject or domesticated animal, wherein choline and succinate are derived from a choline salt and a succinate salt, correspondingly.
2. A composition of clause 1, further comprising nicotinamide, or nicotinamide derivate, wherein the molar ratio choline: succinate; nicotinamide/nicotinamide derivate is from about 2:1:0.01 to about 2:1:10, and wherein the nicotinamide derivate is preferably nicotinamide riboside.
3. A composition of clauses 1 or 2, wherein choline and succinate are derived from di-choline succinate salt.
4. A composition of any of clauses 1 to 3, wherein the amount of nicotinamide, or the nicotinamide derivate, is from around 10 mg to around 4000 mg.
5. A composition of any of clauses 1 to 4, wherein the amount of dicholine succinate salt is from around 10 mg to around 5000 mg.
6. A composition of any of clauses 1 to 5, further comprising at least one vitamin B selected from vitamin B12 (cobalamin or methylcobalamin), vitamin B6 (pyridoxine), and/or vitamin B9 (folic acid or folacin).
7. A composition of any of clauses 1 to 6, further comprising an omega-3 unsaturated acid, such as eicosapentaenoic acid (EPA) and docosahexaenoic acid (DHA), preferably DHA.
8. A composition of any of clauses 1-7, wherein the composition is a nutritional or nutraceutical composition, medical food product or a pharmaceutical composition, or it is comprised in a nutritional or nutraceutical composition medical food product or a pharmaceutical composition.
9. A composition of any of preceding clauses 1-8, wherein the mammal subject is a human subject who is
   (i) an undernourished individual or an individual receiving insufficient amount of folate, and/or choline from the diet;
   (ii) an overweight or obese individual;
   (iii) an individual undergoing or recovering from metabolic, psychological and/or environmental stress;
   (iv) an aging individual;
   (v) a patient undergoing or recovering from a therapeutic treatment;
   (vi) a patient having a chronic, degenerative or inflammatory disease; and/or
   (vii) an individual who is recovering from a surgery or physical trauma.
10. A composition of any of clauses 1 to 9, wherein the human subject is an individual who is suffering or recovering from a disease or disorder selected from a muscle degenerative disease or age-related sarcopenia or muscle-wasting; a lever disease, such as nonalcoholic fatty liver disease (NAFLD) or liver fibrosis; neurodegenerative or neurological disease, such as Alzheimer's, Parkinson's, or Huntington's disease; an inflammatory disease, such as Crohn's disease, inflammatory bowel disease (IBD) or cardiovascular disease (CVD).
11. A composition of any of clauses 1-8, wherein the mammal subject is a pregnant woman or a child.
12. A composition of any of clauses 1-8, wherein the mammal subject is a healthy human individual.
13. A composition of any of clauses 1-8, wherein the mammal is a domestic mammal such as cattle, goat, sheep, pig, horse or alike, or a pet mammal, such as dog, cat, rabbit, or alike.
14. A composition of any of clauses 1-13, wherein the composition is
   for use in treating or preventing metabolic stress, and/or
   for use in reducing catabolism and/or for stimulating anabolism,
   in a subject in need, wherein said subject is a human or domesticated animal subject, which
      Is affected by lack or excess of nutrients in the diet, and/or
      has an increase in energy expenditure due to injury or illness.
15. A method for the dietary management and/or mitigation of the risk of
   catabolism and/or increased energy expenditure due to injury, Illness and/or unbalanced nutrition a mammal subject; and/or
   development of a pathological condition or a symptom associated with an imbalanced, damaged, or reduced level of one-carbon (1C) metabolism; and/or
   occurrence and/or re-occurrence of a symptom or condition associated with an imbalanced, damaged or reduced level of one-carbon (1C) metabolism;
in a mammal subject, comprising administering to said mammal subject at least once a day a composition of any of clauses 1 to 8.
16. A method of clause 15, wherein the mammal subject is a human individual according to any of clauses 9-12, or a mammal animal of embodiment 13.
17. A method of any of clauses 1-16, wherein the composition is administered daily in one or more doses for a period of several days, such as 2-7 days or more.
18. A dietary supplement, food or beverage product comprising dicholine succinate and docosahexaenoic acid (DHA), wherein the amount of dicholine succinate is from around 10 mg to around 5000 mg, and the amount of DHA is from around 200 mg to around 1000 mg.
19. A dietary supplement, food or beverage product of clause 18, further comprising nicotinamide, or a nicotinamide derivate, wherein the molar ratio choline: succinate: nicotinamide, or nicotinamide derivate, is from around 2:1:0.01 to around 2:1:10, and wherein the nicotinamide derivate is nicotinamide riboside.
20. A dietary supplement, food or beverage product of any of clauses 18-19, further comprising at least one vitamin B selected from vitamin B12 (cobalamin or methylcobalamin), vitamin B6 (pyridoxine), and vitamin B9 (folic acid or folacin).
21. A dietary supplement, food or beverage product of any of clauses 18-20, wherein said dietary supplement, food or beverage product support, enhance and restore one carbon metabolism and stimulates anabolism in a human or domesticated animal subject

The subject matter of the disclosure is further illustrated by the non-limiting examples described below.

### Examples

The examples presented below are illustrative examples only and are not intended to be limiting to the scope of the disclosure or claims in any way.

### Example 1. Beverages Comprising Nutritional Compositions of the Disclosure

Beverage-1. The beverage-1 is prepared by dissolving DiSu in 330 ml of water.

Beverage-1

| Ingredient | Content, per serving |
|---|---|
| DiSu | 250 mg |
| gluconic acid | qs to pH 6.5 |
| water | to 330 ml |

Beverage-2. The beverage-2 is prepared by mixing NAM with DiSu in amounts as indicated below and dissolved in 330 ml of water.

Beverage-2

| Ingredient | Content, per serving |
|---|---|
| NAM | 188 mg |
| DiSu | 250 mg |
| gluconic acid | qs to pH 6.5 |
| water | to 330 ml |

The molar ratio choline: succinate: NAM in this example beverage-2 is 2:1:2.

Beverage-3. The beverage-3 is prepared by mixing NAM with DiSu in amounts as indicated below and dissolved in 330 ml of water.

Beverage-3

| Ingredient | Content, per serving |
|---|---|
| NAM | 250 mg |
| DiSu | 500 mg |
| gluconic acid | qs to pH 6.5 |
| water | to 330 ml |

The molar ratio choline: succinate: NAM in this example beverage-3 is 2:1:1.

Beverage-4. The beverage-4 is prepared by mixing NAM with DiSu in amounts as indicated below and dissolved in 500 ml of water.

Beverage-4

| Ingredient | Content, per serving |
|---|---|
| NAM | 210 mg |
| DiSu | 560 mg |
| gluconic acid | qs to pH 6.5 |
| water | to 500 ml |

The molar ratio choline: succinate: NAM in this example beverage-4 is 2:1:1.

Beverage-5. The beverage-5 is prepared by mixing NAM with DiSu in amounts as indicated below and dissolved in 330 ml of water.

Beverage-5

| Ingredient | Content, per serving |
|---|---|
| NAM | 375 mg |
| DiSu | 100 mg |
| gluconic acid | qs to pH 6.5 |
| water | to 330 ml |

The molar ratio choline: succinate: NAM in this example beverage-5 is about 2:1:10.

Beverage-6. The beverage-6 is prepared by mixing NAM with DiSu in amounts as indicated below and dissolved in 330 ml of water.

Beverage-6

| Ingredient | Content, per serving |
|---|---|
| NAM | 7 mg |
| DiSu | 2000 mg |
| succinic acid | qs to pH 6.5 |
| water | to 330 ml |

The molar ratio choline: succinate: NAM in this example beverage-6 is 2:1:0,01.

Beverage-7. The beverage-7 is prepared by mixing NAM with DiSu in amounts as indicated below and dissolved in 330 ml of water to provide a beverage.

Beverage 1

| Ingredient | Content, per serving |
|---|---|
| NAM | 37 mg |
| DiSu | 250 mg |
| gluconic acid | qs to pH 6.5 |
| Water | to 330 ml |

The molar ratio choline: succinate: NAM in this beverage-7 is 2:1:0.4).

### Example 2. Evaluation of the Effect of DiSu on the Production of Betaine In Vitro in Isolated Mitochondria

Livers from male Wistar rats were homogenized using a Teflon®-glass Potter-Elvehjem homogenizer in the isolation medium containing 0.25 M sucrose, 0.02 M Tris-HCl, 0.001 M EDTA, pH 7.2. The homogenates prepared in this way were centrifuged at 600 g for 10 min at 4° C. to precipitate nuclei and cell debris. Then the supernatant was separated from the sediment and subjected to centrifugation at 8500 g for 10 min at 4° C. to precipitate mitochondria. Isolated mitochondria were incubated for 15 min at 37° C. in a medium containing 35 mM Tris-HCl (pH 7.6) with or without a test substrate (choline chloride or DISU), after which the concentration of betaine (N,N,N-trimethylglycine) in the incubation medium was determined using LC-MS/MS.

Results: Incubation of isolated mitochondria in the presence of choline succinate salt 2:1 leads to higher betaine yields compared to incubation in the presence of choline chloride.

### Example 3. Impact of Intake of Three Different Choline Supplements on Plasma Concentration and Kinetics of Choline, Betaine in Adult Human Subjects

Study: a randomized cross-over study on 12 adult male subjects.

Subjects: 12 healthy adult male subjects (3 placebo and 9 test supplement (each supplement group-3 subjects)) who are between 30 and 60 years of age.

Duration: One-day oral administration of a choline supplement was followed by 1 week of wash-out before the start of administration of the next (different) choline supplement. Total duration of the study is 24 days.

Exclusion criteria: Alcohol abuse, acute illness, chronic diseases (like diabetes, metabolic syndrome, thyroid diseases, pancreas insufficiency), intake of choline-containing nutritional supplements, or missing consent.

The test subjects were to drink the adequate intake (AI) of choline according to NAM (550 mg/d) (Evaluation of Dietary Reference Intakes and its Panel on Folate, Other B Vitamins, and Choline (1998) Dietary Reference Intakes for Thiamin, Riboflavin, Niacin, Vitamin B6, Folate, Vitamin B12, Pantothenic Acid, Biotin, and Choline. National Academies Press (US), Washington (DC). PMID: 23193625) in the form of form of beverage comprising:
740 mg choline chloride
1336 mg choline bitartrate
855 mg dicholine succinate

Choline chloride (CAS: 67-48-1), choline bitartrate (CAS: 87-67-2), di-choline succinate (CAS: 109438-15-5). All have GRAS (Generally Recognized As Safe) status.

Subjects took each of the three supplements in randomized sequence with interspersed wash-out periods of 1 week. Substances were dissolved (choline chloride, choline bitartrate, dicholine succinate) in 330 ml beverage (50% v/v water and 50% v/v a blend of apple, orange, carrot and ginger juice). Placebo group subjects were to take the same beverage that did not comprise the supplements. The beverages were consumed in the morning after overnight fasting. Blood (2.7 ml EDTA) was taken before intake (−0.1 h) and at 0.5, 1, 1.5, 2, 3, 4, 5, and 6 h after intake. Collected blood was immediately centrifuged at 1000×g at room temperature for 10 min. Plasma was separated and stored at −80° C. until analysis. Plasma samples were processed according to established standard procedures (Bernhard W, Raith M, Kunze R, Koch V, Heni M, Maas C, Abele H, Poets C F, Franz A R (2015) Choline concentrations are lower in postnatal plasma of preterm infants than in cord plasma. Eur. J. Nutr. 54 (5): 733-741. https://doi.org/10.1007/s00394-014-0751-7; Bernhard W, Full A, Arand J, Maas C, Poets C F, Franz A R (2013) Choline supply of preterm infants: assessment of dietary intake and pathophysiological considerations. Eur. J. Nutr. 52 (3): 1269-1278. https://doi.org/10.1007/s00394-012-0438-x). Equipment for analysis: TSQ Quantum Discovery MAX tandem mass spectrometer, a Finnigan Surveyor Autosampler Plus, and a Finnigan Surveyor MS Pump Plus (Thermo Fisher Scientific, Dreieich, Germany), and choline, betaine were separated on a Polaris Si-A® column at 40° C. and the components were analyzed.

Results: There was no difference between the area under the curve (AUC) at 0-6 h for choline plasma concentrations after administration of the three different choline supplements. The AUC (0-6 h) of betaine concentrations had the highest values for di-choline succinate and lowest for choline chloride (dicholine succinate>choline bitartrate>choline chloride).

Conclusion: All tested supplements increased choline and betaine plasma concentrations with similar kinetics, yet a difference between supplements were observed, in particular, dicholine succinate supplementation led to better values for both absorption kinetic of choline and kinetic of transformation of choline to betaine.

The observed higher increase in plasma betaine than that of choline suggests that feeding the one-carbon pool via betaine is an important aspect of any choline supplementation, and supplementation with dicholine succinate is more advantageous in this respect compared with other tested choline supplements.

### Example 4. Bioenergetics, One-Carbon Metabolism, and Muscle Function Improvement in Elderly Subjects Treated with Beverage Comprising DiSu

Study: a randomized, double-blind, single-center, placebo-controlled human study enrolling 66 healthy elderly subjects (33 placebo and 33 beverage administration) who are ≥65 and ≤90 years of age, BMI between 18-32. DISU-containing beverage (choline supplement) or placebo will be orally administered for 4 months.
Allocation: Randomized
Intervention Model: Parallel Assignment
Masking: Triple (Participant, Investigator, Outcomes Assessor)
Active Comparator: Dietary Choline and Nicotinamide Supplement (Example 1, Beverage 7)
   1 dose of Test beverage to be taken daily.
Placebo Comparator: Dietary Supplement: Placebo beverage (excipients containing beverage)
   1 dose of Placebo beverage to be taken daily

Inclusion Criteria: Adult human volunteers of all sexes≥65 and ≤90 years of age; Capable of walking 6 minute walk distance of <550 meters; Have ATP max<1 mM/sec (in the hand FDI muscle);

Exclusion Criteria: significant disease(s) or condition(s), hospitalization within 3 months for major atherosclerotic events (defined as combined incidence of myocardial infarction, urgent target-vessel revascularization, coronary bypass surgery and stroke, and for any hospitalization within 2 months; have any implants, including cardiac pacemaker; chronic, uncontrolled hypertension (i.e., Baseline SBP>150 mm Hg, DBP 22 90 mm Hg) or a SBP>150 mm Hg or DBP>95 mm Hg at the time of screening or baseline. If the initial BP reading is above these values, the reading may be repeated one time within 20 minutes of the initial reading; clinically significant abnormalities on physical examination; clinically significant and chronic uncontrolled renal, hepatic, pulmonary, endocrine, neurologic disorders, bone, or gastrointestinal system dysfunction; history of seizures or epilepsy; history of serious mental illness; suspicion, or recent history, of alcohol or substance abuse or tobacco use.

### Primary Outcome Measures:

1. Change in 6 minute walking distance (6MWD) at the end of study intervention compared to baseline [Time Frame: 4 months].
2. Percent change from baseline in ATP max (maximal ATP synthesis rate) in hand skeletal muscle (via Magnetic Resonance Spectroscopy) [Time Frame: 2,4 months].
3. Change in plasma levels of choline, betaine and homocysteine [Time Frame: 2,4-weeks].

### Secondary Outcome Measures:

1. Percent change from baseline in contraction number during a hand muscle fatigue test [Time Frame: 2,4 months].
2. Percent change from baseline in ATP max (maximum ATP synthesis rate) in leg skeletal muscle (via MRS) [Time Frame: 4 months].
3. Percent change from baseline in contraction number during a leg muscle fatigue test [Time Frame: 4 months].
4. Change in Short Physical Performance Battery (SPPB) scores at the end of study intervention compared to baseline [Time Frame: 4 months].
5. Change in exercise tolerance compared to baseline (via cycle ergometry) [Time Frame: 4 months].
6. Change in hand grip strength at the end of study intervention compared to baseline [Time Frame: 4 months].
7. Change in leg muscle strength (1-RM and 10-RM) at the end of study intervention compared to baseline [Time Frame: 4 months].
8. Change in muscle size (cross-sectional area of the muscles) at the end of study intervention compared to baseline [Time Frame: 4 months].
9. Change in mitochondrial function on muscle biopsy samples at the end of study intervention compared to baseline (via respirometry) [Time Frame: 4 months].
10. Change from baseline in plasma lipid profile [Time Frame: 4 months].

1-21. (canceled)

22. A method for reducing metabolic stress in a subject experiencing metabolic stress, the method comprising:
providing for consumption by the subject, a composition formulated to enhance one-carbon metabolism and containing choline and succinate in a molar ratio of choline to succinate of 2:1 in a daily dosage effective to reduce catabolism and/or promoting anabolism in the subject consuming the daily dosage during an initial dosage period.

23. The method of claim 22, further comprising formulating the choline and the succinate in the composition to be in the form of dicholine succinate (DiSu).

24. The method of claim 22, further comprising formulating the daily dosage of DiSu in the composition to be within a range of from about 100 mg to about 2000 mg per serving in one or more servings up to a total of about 5000 mg per day.

25. The method of claim 22, further comprising, formulating the composition to further include a nicotinamide component selected from nicotinamide, nicotinamide riboside, nicotinamide mononucleotide, and combinations thereof, wherein the molar ratio of the choline to the succinate to the nicotinamide component is 2:1:0.01-10.

26. The method of claim 25, further comprising, formulating relative amounts of the choline, succinate, and nicotinamide components in the daily dosage to concurrently:
enhance cell energy metabolism in the subject by synergistically increasing availability of adenosine triphosphate (ATP) and nicotinamide adenine dinucleotide (NAD); and
enhance 1C metabolism in the subject by synergistically increasing availability of S-adenosylmethionine (SAMe) and decreasing a level of homocysteine.

27. The method of claim 22, further comprising, formulating the composition to further include a daily dosage of docosahexaenoic acid (DHA) of from about 200 mg to about 1000 mg.

28. The method of claim 22, further comprising, formulating the composition to further include one or more of cobalamin, pyridoxine, and folacin.

29. The method of claim 22, further comprising, formulating the daily dosage of the composition for consumption by the subject where the subject is a domesticated mammal rather than a human subject.

30. The method of claim 22, further comprising, providing the composition in a dosage form sufficient to provide the daily dosage where the initial dosage period is at least 7 days.

31. A composition comprising:
a combination of choline and succinate in a molar ratio of choline to succinate of 2:1, the combination of choline and succinate formulated to include a daily dosage of the combination of choline and succinate that is effective to enhance one-carbon (1C) metabolism in a subject consuming the composition during an initial dosage period.

32. The composition of claim 31, wherein the combination of the choline and succinate comprises dicholine succinate (DiSu).

33. The composition of claim 32, further comprising a dosage of the composition that is effective in the subject consuming the composition during an initial dosage period for:
increasing a plasma level of betaine;
increasing a plasma level of S-adenosylmethionine; and
decreasing a plasma level of homocysteine.

34. The composition of claim 33, further comprising a nicotinamide component selected from nicotinamide, nicotinamide riboside, nicotinamide mononucleotide, and combinations thereof, wherein the molar ratio of the choline to the succinate to the nicotinamide component is 2:1:0.01-10.

35. The composition of claim 34, further comprising an effective amount of an omega-3 unsaturated acid that when added in as a further component of the composition produces synergistic effects relative to a sum of effects of said choline, succinate, and nicotinamide components of the composition on the 1C metabolism of the subject consuming the composition.

36. The composition of claim 35, wherein the omega-3 unsaturated acid is docosahexaenoic acid (DHA).

37. The composition of claim 32, further comprising one or more of cobalamin, pyridoxine, and folacin.

38. The composition of claim 33, wherein the daily dosage of DiSu in the composition is within a range of from about 100 mg to about 1000 mg per serving in one or more servings up to a total of about 5000 mg per day.

39. A method for dietary management of metabolic stress and/or unbalanced 1C metabolism one-carbon (1C) in a mammal, the method comprising:
providing for consumption by a subject, a nutritional supplement comprising choline and succinate in a molar ratio of choline to succinate of 2:1 in a daily dosage effective to reduce catabolism and/or promoting anabolism in the subject consuming the daily dosage during an initial dosage period.

40. The method of claim 39, further comprising formulating the choline and succinate in the form of dicholine succinate DiSu with the daily dosage in a range of from about 100 mg to about 2000 mg per serving in one or more servings up to a total of about 5000 mg per day.

41. The method of claim 40, further comprising, formulating relative amounts of the choline, succinate, and nicotinamide components in the daily dosage to concurrently:
enhance cell energy metabolism in the subject by synergistically increasing availability of adenosine triphosphate (ATP) and nicotinamide adenine dinucleotide (NAD); and
enhance 1C metabolism in the subject by synergistically increasing availability of betaine, S-adenosylmethionine (SAMe) and decreasing a level of homocysteine.

42. The method of claim 39, further comprising formulating the nutritional supplement to further include a daily dosage of docosahexaenoic acid (DHA) of from about 200 mg to about 1000 mg.

43. The method of claim 39, further comprising, formulating the nutritional supplement to further include one or more of cobalamin, pyridoxine, and folacin.
